(19) United States
(12) Patent Application Publication
(10) Pub. No.: US 2015/0167456 A1
(43) Pub. Date: Jun. 18, 2015

(54) METHODS OF PREDICTING A RESERVOIR FLUID BEHAVIOR USING AN EQUATION OF STATE

(71) Applicant: Cyrus A. Irani, Houston, TX (US)

(72) Inventor: Cyrus A. Irani, Houston, TX (US)

(73) Assignee: HALLIBURTON ENERGY, INC., Houston, TX (US)

(21) Appl. No.: 14/405,249

(22) PCT Filed: Aug. 7, 2012
(86) PCT No.: PCT/US12/49861

§ 371 Date: Dec. 3, 2014

(51) Int. Cl. E21B 49/08; G01N 33/28

(52) U.S. Cl. CPC E21B 49/08; G01N 33/28

(57) ABSTRACT

A method of using data obtained from a sample of a reservoir fluid comprises: collecting the sample in a sample container, wherein the sample container includes a sample receptacle, and wherein the step of collecting comprises allowing or causing the sample to flow into the sample receptacle; determining at least one compositional component of the sample using an analyzer, wherein the step of determining is performed during the step of collecting; and using an equation of state to predict a potential change in at least one property of the reservoir fluid based on the determination of the at least one compositional component of the sample. Another method comprises: transferring the sample from the sample container to a second container, wherein the step of determining is performed after the step of collecting and during fluid flow of the sample.

### TECHNICAL FIELD

Methods of using data obtained from a sample of a reservoir fluid to obtain predictive property changes to the fluid over the life of a well are provided. The data obtained can be one or more compositional components of the reservoir fluid sample. The data can be obtained via the use of an analyzer. The one or more compositional components can then be input into an equation of state in order to predict the reservoir fluid behavior at varying temperatures and pressures.

### SUMMARY

According to an embodiment, a method of using data obtained from a sample of a reservoir fluid comprises: collecting the sample in a sample container, wherein the sample container includes a sample receptacle, and wherein the step of collecting comprises allowing or causing the sample to flow into the sample receptacle; determining at least one compositional component of the sample using an analyzer, wherein the step of determining is performed during the step of collecting; and using an equation of state to predict a potential change in at least one property of the reservoir fluid based on the determination of the at least one compositional component of the sample.

According to another embodiment, a method of using data obtained from a sample of a reservoir fluid comprises: collecting the sample in a sample container; transferring the sample from the sample container to a second container, wherein the step of transferring is performed after the step of collecting; determining at least one compositional component of the sample using an analyzer, wherein the step of determining is performed after the step of collecting; and using an equation of state to predict a potential change in at least one property of the reservoir fluid based on the determination of the at least one compositional component of the sample.

### BRIEF DESCRIPTION OF THE FIGURES

The features and advantages of certain embodiments will be more readily appreciated when considered in conjunction with the accompanying figures. The figures are not to be construed as limiting any of the preferred embodiments.

FIG. 1 is a diagram of a sample container including a sample receptacle.

FIG. 2 is a diagram of an analyzer for analyzing one or more compositional components of a sample.

FIG. 3 is a diagram of the analyzer from FIG. 2 according to an embodiment depicting analysis of the sample during collection of the sample.

FIG. 4 is a diagram of the analyzer from FIG. 2 according to another embodiment depicting analysis of the sample during transference of the sample.

### DETAILED DESCRIPTION

As used herein, the words “comprise,” “have,” “include,” and all grammatical variations thereof are each intended to have an open, non-limiting meaning that does not exclude additional elements or steps.

It should be understood that, as used herein, “first,” “second,” “third,” etc., are arbitrarily assigned and are merely intended to differentiate between two or more analyzers, heating elements, etc., as the case may be, and does not indicate any sequence. Furthermore, it is to be understood that the mere use of the term “first” does not require that there be any “second,” and the mere use of the term “second” does not require that there be any “third,” etc.

As used herein, a “fluid” is a substance having a continuous phase that tends to flow and to conform to the outline of its container when the substance is tested at a temperature of 71° F. (22° C.) and a pressure of one atmosphere “atm” (0.1 megapascals “MPa”). A fluid can be a liquid or gas. A fluid can have only one phase or more than one phase. In the oil and gas industry, a fluid having only one phase is commonly referred to as a single-phase fluid and a fluid having more than one phase is commonly referred to as a multi-phase fluid. A colloid is an example of a multi-phase fluid. A colloid can be: a slurry, which includes a continuous liquid phase and undissolved solid particles as the dispersed phase; an emulsion, which includes a continuous liquid phase and at least one dispersed phase of immiscible liquid droplets; a foam, which includes a continuous liquid phase and a gas as the dispersed phase; or a mist, which includes a continuous gas phase and liquid droplets as the dispersed phase.

Oil and gas hydrocarbons are naturally occurring in some subterranean formations. A subterranean formation containing oil or gas is sometimes referred to as a reservoir. A reservoir may be located under land or off shore. In order to produce oil or gas, a wellbore is drilled into a reservoir or adjacent to a reservoir.

A well can include, without limitation, an oil, gas, or water production well, or an injection well. As used herein, a “well” includes at least one wellbore. A wellbore can include vertical, inclined, and horizontal portions, and it can be straight, curved, or branched. As used herein, the term “wellbore” includes any cased, and any uncased, open-hole portion of the wellbore. A near-wellbore region is the subterranean material and rock of the subterranean formation surrounding the wellbore. As used herein, a “well” also includes the near-wellbore region. As used herein, the phrase “into a well” includes into any portion of the wellbore or into the near wellbore region via the wellbore.

It is often desirable to take a sample of a reservoir fluid. There are a variety of instruments that can be used to collect a sample of reservoir fluids. One such instrument is the ARMADA® sampling system, marketed by Halliburton Energy Services, Inc. In order to collect a sample, the sampling system is placed into a wellbore at a desired location. The sampling system functions to collect multiple samples of the reservoir fluid at that location. The ARMADA® sampling system is currently able to collect up to nine unique samples of the reservoir fluid per trip. The sampling system is then returned to the surface where the samples can be retrieved from the system.

In the oil and gas industry it is often desirable to analyze a sample of a reservoir fluid. The sample can be analyzed to determine, for example, the composition of the sample. If the sample contains corrosive substances, then the fluid might be detrimental to wellbore operations, for example, harmful to wellbore equipment, such as pumping equipment or pipes. Examples of corrosive substances include, but are not limited to, high amounts of an acid gas, such as carbon dioxide gas (acid gas wells) and wells containing high amounts of a sour gas, such as hydrogen sulfide gas (sour gas wells).

Another potentially detrimental substance is an asphaltene. If asphaltenes are present in the reservoir fluid, then generally they are in solution due to being stabilized by resins. However, if the relative resin content decreases, then the asphaltenes may precipitate out of the fluid and deposit on pipe walls, restricting or interrupting fluid flow. It is relatively costly to remove such asphalt deposits, which may require grinding or scraping operations for removal. Other potentially detrimental substances are aromatics and naphthanates. When combined with water, aromatics and naphthanates can cause foaming of the solution, somewhat like when water is combined with soap. The foam can also restrict or interrupt fluid flow.

Another potentially detrimental substance is a gas hydrate. Generally, a substance containing between one and six carbon atoms (C₁ to C₆) is a gas at wellbore temperatures and pressures. However, during wellbore operations, depending on the temperature at the wellhead, some or all of the gas may form gas hydrates. Gas hydrates occur naturally onshore in permafrost regions, and at certain depths in the sea where water and gas combine at low temperatures and high pressures to form the hydrate. Methane (C₁), or natural gas, is typically the dominant gas in the hydrate structure. As gas emerges from the wellhead, water molecules from the surrounding environment form a cage-like structure around high concentrations of the gas molecules and freeze into a solid gas/water structure. If a sufficient amount of gas hydrates form, the hydrates can block or clog valves and pipes leading to the surface from the cap. As such it may be desirable to test a reservoir fluid for its gas-to-oil (GOR) ratio. This ratio, along with the temperature at the wellhead, can be useful in predicting the likelihood of gas hydrate formation. Therefore, by determining the composition of a reservoir sample, one can determine the exact substances, such as detrimental substances, that make up the sample.

There are several devices that can be used to analyze a fluid and determine the composition of the fluid. Some devices are designed to be used in a laboratory setting and other devices can be used in a well or at or near the well site. A spectrometer is an example of a device that can be used to analyze a fluid. Spectroscopy is the study of the interaction between matter and radiated energy. Generally, an energy source, such as light, is directed onto and possibly through a sample. A detector can then detect the light emitted from the source after the light passes through the sample. One of the central concepts in spectroscopy is a resonance and its corresponding resonant frequency. Spectroscopic data is often represented by a spectrum, a plot of the response of interest as a function of wavelength or frequency. A plot of amplitude versus excitation frequency will have a peak centered at the resonance frequency. This plot is one type of spectrum, with the peak often referred to as a spectral line, and most spectral lines have a similar appearance.

Spectroscopy can be classified based on the type of the radiative energy source, the nature of the interaction, or the type of material of the sample. The types of radiative energy can include electromagnetic radiation, particles, acoustic, and mechanical. Techniques that employ electromagnetic radiation are typically classified by the wavelength region of the spectrum and include microwave, terahertz, far infrared, infrared, near infrared, visible, ultraviolet, x-ray and gamma spectroscopy. A wavelength is the distance over which a wave repeats itself, is inversely proportional to the frequency, and is reported in units of length (e.g., micrometers, nanometers, or meters). The higher the frequency the shorter the wavelength and the lower the frequency the longer the wavelength. The frequency is the number of occurrences per unit of time, reported in units of seconds. A wavenumber is proportional to the reciprocal of the wavelength, reported in units of inverse meters (m⁻¹) or inverse centimeters (cm⁻¹). The wavelength regions for each type of electromagnetic radiation are different. For example, the near infrared region has a wavelength of approximately 800 nanometers (nm) to 2500 nm; whereas, the ultraviolet region has a wavelength of approximately 10 nm to 400 nm. Uncharged and charged particles, due to their de Broglie wavelength, can also be a source of radiative energy and electrons, protons, and neutrons are commonly used. For a particle, its kinetic energy determines its wavelength. Acoustic spectroscopy involves the use of radiated pressure waves. Mechanical methods can be employed to impart radiating energy, similar to acoustic waves, to solid materials.

Types of spectroscopy can also be distinguished by the nature of the interaction between the energy and the material. These interactions include absorption, emission, elastic scattering and reflection, impedance, inelastic scattering, and coherent interactions. Absorption occurs when energy from the radiative source is absorbed by the material. Absorption is often determined by measuring the fraction of energy transmitted through the material, wherein absorption will decrease the transmitted portion. Emission indicates that radiative energy is released by the material. A material's blackbody spectrum is a spontaneous emission spectrum determined by its temperature. Emission can be induced by electromagnetic radiation in the case of fluorescence. Elastic scattering and reflection spectroscopy determine how incident radiation is reflected or scattered by a material. Impedance spectroscopy studies the ability of a medium to impede or slow the transmittance of energy. Inelastic scattering involves an exchange of energy between the radiation and the matter that shifts the wavelength of the scattered radiation. These include Raman and Compton scattering. Coherent or resonance spectroscopies are techniques where the radiative energy couples two quantum states of the material in a coherent interaction that is sustained by the radiating field. The coherence can be disrupted by other interactions, such as particle collisions and energy transfer, and thus, often require high intensity radiation to be sustained. Nuclear magnetic resonance (NMR) spectroscopy is a widely used resonance method and ultrafast laser methods are also now possible in the infrared and visible spectral regions.

Another example of an analyzer that can be used to analyze a fluid is a multivariate optical element (MOE) calculation device. The MOE calculation device is described fully in U.S. Pat. No. 7,697,141 B2, issued on Apr. 13, 2010 to Jones, et al., which is hereby incorporated by reference in its entirety for all purposes. If there is any conflict in the usages of a word or term in this specification and one or more patents or other documents that may be incorporated herein by reference, then the definitions that are consistent with this specification control and should be adopted.

A MOE calculation device can include: a light source; a multivariate optical element (MOE), which is an optical regression calculation device; a detector for detecting light reflected from MOE; and a detector for detecting the light transmitted by MOE. The MOE is a unique optical calculation device that comprises multiple layers. The multiple layers can have different refractive indices. By properly selecting the materials of the layers and their spacing, the MOE calculation device can be made to selectively pass predetermined fractions of light at different wavelengths. Each wavelength is given a predetermined weighting or loading factor. The thicknesses and spacing of the layers may be determined using a variety of approximation methods from the spectrograph of the property of interest. The approximation methods may include inverse Fourier transform (IFT) of the optical transmission spectrum and structuring the optical calculation device as the physical representation of the IFT. The approximations convert the IFT into a structure based on known materials with constant refractive indices.

The weightings that the MOE layers apply at each wavelength are set to the regression weightings described with respect to a known equation, or data, or spectral signature which can be found for the given property of interest. The optical calculation device MOE performs the dot product of the input light beam into the optical calculation device and a desired loaded regression vector represented by each layer for each wavelength. The MOE output light intensity is directly related to, and is proportional to, the desired sample property. The output intensity represents the summation of all of the dot products of the passed wavelengths and corresponding vectors.

By way of example, if the property of interest is resin in a reservoir fluid sample, and the regression vectors are that of the resin, then the intensity of the light output of the MOE is proportional to the amount of resin in the sample through which the light beam input to the optical calculation device has either passed or has been reflected from or otherwise interacted with. The ensemble of layers corresponds to the signature of resin. These wavelengths are weighted proportionately by the construct of the corresponding optical calculation device layers. The resulting layers together produce an optical calculation device MOE output light intensity from the input beam. The output light intensity represents a summation of all of the wavelengths, dot products, and the loaded vectors of that property, e.g., resin. The output optical calculation device's intensity value is proportional to the amount of resin in the sample being analyzed. In this manner, a MOE calculation device is produced for each property to be determined in the sample.

It can be desirable to determine the equation of state of a reservoir fluid. As used herein, an “equation of state” (EOS) is a thermodynamic equation or formula that describes the state of matter under a given set of physical conditions and provides a mathematical relationship between two or more state functions associated with the matter. Commonly, the state functions include pressure, volume and temperature. In a subterranean formation, the temperature will generally remain constant; however, the pressure of the formation can often change during the course of oil or gas operations. If a plot of pressure versus volume for a given substance at a constant temperature is obtained, the EOS can represent the volumetric behavior of the pure substance in the entire range of volume, both in the liquid and gaseous states. As such, the EOS can represent the phase behavior of the substance: in the two-phase envelope (i.e., inside the binodal curve); on the two-phase envelope; and outside the two-phase envelope.

It is not uncommon for the life of a well (i.e., the time the well is operational) to range from a few years to more than 10 years. As operations occur and continue, it is common for some properties of a reservoir fluid to change. Such changes can include the percentage of liquid to gas and other properties of the fluid. The EOS for a specific sample of a reservoir fluid can be used to determine what changes are likely to occur to the reservoir fluid over the life of the well. This predictive information can be quite valuable to the reservoir engineer in charge of field operations, who can then make decisions necessary for optimizing the field's behavior based on the information.

There are several different equations of state. EOSs can be divided into two main groups, cubic and non-cubic. Some examples of EOS formulas include, but are not limited to, Boyle, Van der Waals, Redlich-Kwong (RK), Soave-Redlich-Kwong (SRK), Peng-Robinson (PR), Peng-Robinson-Stryjek-Vera (PRSV), Patel-Teja (PT), Schmit-Wenzel (SW), and Esmaeilzadeh-Roshanfekr (ER). Certain EOS formulas can be more predictive compared to other EOS formulas. For example, research has shown that non-cubic equations can better describe the volumetric behavior of pure substances, but may not be the best equations for complex hydrocarbon mixtures, such as reservoir fluids. Moreover, a single EOS may not be able to predict all the thermodynamic properties of different kinds of reservoir fluids. As such, the EOS may need to be selected based on the exact composition of the fluid.

The predictive information obtained from a particular EOS can be improved by fine tuning the EOS. A set of physical tests for tuning purposes can be performed on a formation sample. The data obtained from these tests can then be used to fine tune the EOS. Two such tuning tests are a single stage flash (SSF) test and a constant composition expansion (CCE) test. A SSF delivers compositional data about the sample by delivering a flashed gas and a dead oil which can be analyzed, along with the ratio of gas to oil. The dead oil can be further subject to additional measurements such as density to deliver an attribute referred to as API gravity. A CCE study delivers a saturation pressure of the hydrocarbon sample. The saturation pressure can be either a bubble point or a dew point pressure depending on the nature of the hydrocarbon system under analysis. At pressures above the saturation pressure, a CCE study also measures the compressibility of a sample in a single phase state by measuring the change in volume of the sample as the pressure changes. At pressures below the saturation pressure such volumetric measurements are used to determine the relative volumes of the two phases in equilibrium. All of these studies provide invaluable data that can be used to further tune an EOS and thus improve the accuracy of the EOS's predictions.

A need exists to determine the composition of a reservoir fluid sample using an analyzer and then determine the anticipated changes in the properties of the fluid over the life of the well using one or more EOS formulas. By being able to determine the anticipated changes to the properties of the fluid, operation engineers can modify oil or gas techniques in order to optimize the performance and production of the hydrocarbon reserve.

According to an embodiment, a method of using data obtained from a sample of a reservoir fluid comprises: collecting the sample in a sample container, wherein the sample container includes a sample receptacle, and wherein the step of collecting comprises allowing or causing the sample to flow into the sample receptacle; determining at least one compositional component of the sample using an analyzer, wherein the step of determining is performed during the step of collecting; and using an equation of state to predict a potential change in at least one property of the reservoir fluid based on the determination of the at least one compositional component of the sample.

According to another embodiment, a method of using data obtained from a sample of a reservoir fluid comprises: collecting the sample in a sample container; transferring the sample from the sample container to a second container, wherein the step of transferring is performed after the step of collecting; determining at least one compositional component of the sample using an analyzer, wherein the step of determining is performed after the step of collecting; and using an equation of state to predict a potential change in at least one property of the reservoir fluid based on the determination of the at least one compositional component of the sample.

Any discussion of the embodiments regarding the analysis of the sample is intended to apply to all of the method embodiments. Any discussion of a particular component of an embodiment (e.g., a heating element) is meant to include the singular form of the component and also the plural form of the component, without the need to continually refer to the component in both the singular and plural form throughout. For example, if a discussion involves “the heating element 90,” it is to be understood that the discussion pertains to one heating element (singular) and two or more heating elements (plural).

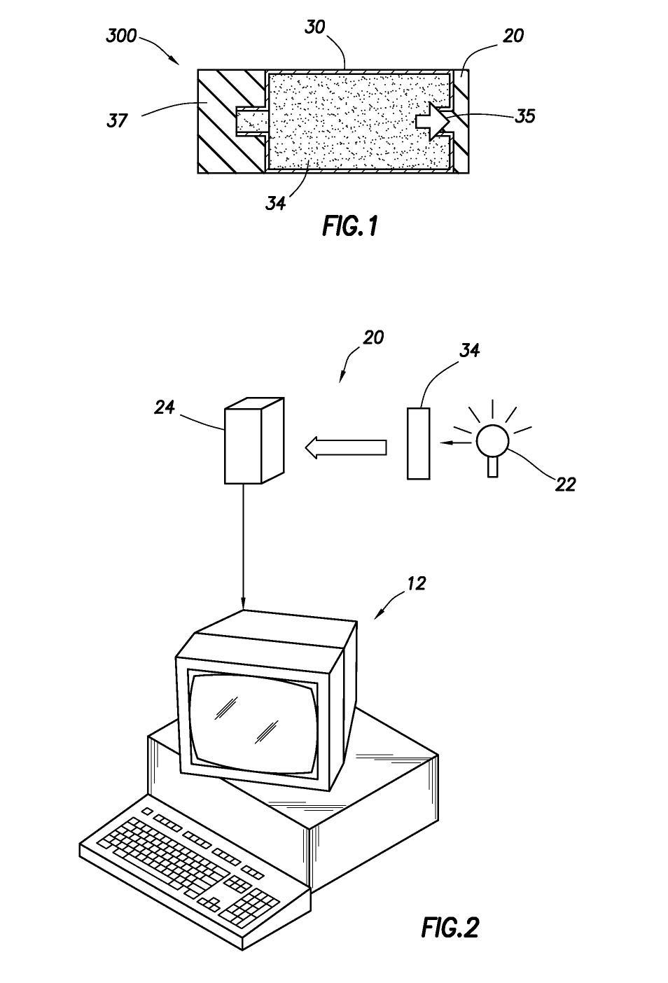

Turning to the Figures. FIG. 1 depicts a sample container 300. The methods include the step of collecting a sample in the sample container 300. According to an embodiment, the sample container 300 is part of the ARMADA® sampling system, marketed by Halliburton Energy Services, Inc. The sample container 300 includes a sample receptacle 30. The sample receptacle 30 can have two ends; a first end and a second end. The sample receptacle 30 can include a first opening. The sample receptacle 30 can also include a second opening. The openings can be located at the first and second ends. The sample receptacle 30 can contain the sample 34. The sample 34 can be collected in the sample container 300 by introducing the sample 34 into the sample receptacle 30 via the first and/or second openings. The sample 34 is a reservoir fluid. The sample 34 can be a substance, such as a solid, liquid, gas, or combinations thereof. The sample 34 can be a single phase fluid or a multi-phase fluid. For example, the sample can be a slurry, emulsion, foam, or mist.

The sample container 300 can further comprise a valve 35. The valve 35 can be a one-way valve. As used herein, the term “one-way valve” means a device that allows a fluid to enter a space within an enclosed area in one direction, but does not independently allow the fluid to exit the space in a reverse direction. Of course, a one-way valve may have a release mechanism whereby a person can externally activate the mechanism thereby causing at least some of the fluid within the sample retaining space to flow out of the enclosed area. However, the one-way valve should be designed such that any fluid that enters the space will not freely flow back out of that space without external intervention. As can be seen in FIG. 1, the valve 35 can be positioned in a first opening of the sample receptacle 30. More than one valve 35 can be located in the sample receptacle 30. According to an embodiment, the step of collecting comprises allowing or causing the sample 34 to flow into the sample receptacle 30. The sample can be introduced into the sample receptacle 30 via the valve 35 positioned in the first opening of the sample receptacle 30. In this manner, the sample can be contained inside the sample receptacle 30 until such time when it is desirable to remove the sample from the sample receptacle 30. The sample container 300 can further include a pressurization compartment (not shown). The pressurization compartment can be used to help maintain the sample 34 in a single phase.

The sample container 300 can further comprise at least one seal 37. The seal 37 can be positioned adjacent to the sample receptacle 30. The seal 37 can be positioned at either end of the sample receptacle 30. The sample container 300 can also include two or more seals. One seal 37 can be positioned at the first end of the sample receptacle 30 and the other seal (not shown) can be positioned at the second end of the sample receptacle 30. According to an embodiment, the seal is designed such that once in place, a sample 34 located within the sample receptacle 30 is not capable of independently exiting the sample receptacle 30. By including two or more seals, any sample 34 located within the sample receptacle 30 can be contained.

The seal 37 can be permanently or removably attached to the sample container 300. By way of example, the seal 37 can be removably attached to the sample receptacle 30. In this manner, once a sample has been collected and is located inside the sample receptacle 30, the sample can be contained within the sample receptacle 30 by attaching the seal 37 to an end of the sample receptacle 30. Moreover, in the event it is desirable to remove the sample 34 from the sample receptacle 30, then the seal 37 can be removed. The seal 37 can include an opening.

The methods include the step of collecting the sample 34 in the sample container 300. The step of collecting can include placing the sample container 300 into a well. The step of collecting can comprise allowing or causing the sample to flow into the sample receptacle 30. The methods can further include the step of removing the sample container 300 from the well, wherein the step of removing can be performed after the step of collecting. By way of example, once the sample 34 is collected, the sample container 300 can be returned to the surface. The methods can further include the step of retrieving the sample receptacle 30 from the sample container 300, wherein the step of retrieving is performed after the step of collecting and/or after the step of removing. The methods can further include the step of attaching one or more seals 37 to the ends of the sample receptacle 30, wherein the step of attaching is performed after the step of retrieving. In this manner, the sample 34 can be contained within the sample receptacle 30. The sample 34 can then be stored, analyzed, transferred, or transported to an off-site location.

The methods include the step of determining at least one compositional component of the sample 34 using an analyzer. The at least one compositional component can be selected from the group consisting of: asphaltenes; saturates; resins; aromatics; solid particulate content; hydrocarbon composition and content; gas composition C₁-C₁₃and content; carbon dioxide gas; hydrogen sulfide gas; total stream percentage of water, gas, oil, and solid particles; water elements including ion composition and content, anions, cations, salinity, organics, contamination; or other hydrocarbon, gas, solids, or water properties that can be related to spectral characteristics, including the use of regression methods. According to an embodiment, two or more compositional components of the sample are determined. Preferably, the number of compositional components of the sample determined is in the range of about 6 to greater than 20. According to another embodiment, at least a sufficient number of compositional components are determined such that the exact equation of state (EOS) to be used can be selected. By way of example, the greater number of compositional components of the sample that are determined, the greater the likelihood one may chose a particular EOS over other equations of state. According to yet another embodiment, at least a sufficient number of compositional components are determined such that the equation of state (EOS) used can yield as accurate predictions as possible. By way of example, the greater number of compositional components of the sample that are determined, the greater the likelihood that the EOS used will yield more accurate predictions compared to when fewer compositional components are determined. According to yet another embodiment, the entire compositional components of the sample are determined.

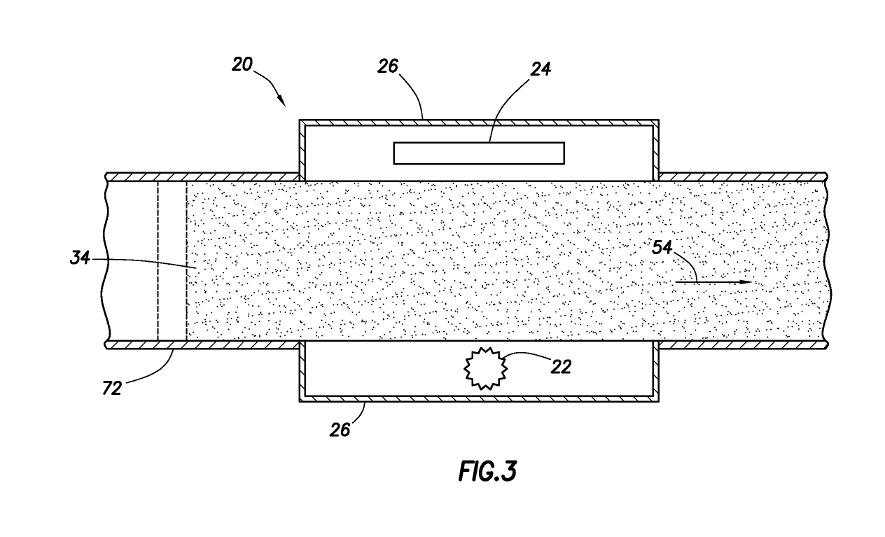

Turning to FIG. 2, the at least one compositional component is determined using an analyzer 20. The analyzer 20 may be an optical analyzer, such as a spectrometer. According to an embodiment, the analyzer 20 includes a source of radiated energy 22 and a detector 24. The source of radiated energy 22 can be a light source. The source of radiated energy 22 and the detector 24 may be selected from all available spectroscopy technologies. The analyzer 20 can also be a multivariate optical element (MOE) calculation device. The MOE calculation device can comprise: the source of radiated energy 22; a multivariate optical element (MOE) (not shown), which is an optical regression calculation device; a first detector for detecting light reflected from MOE; and a second detector for detecting the light transmitted by MOE.

Any available spectroscopy method can be used in the determination of the at least one compositional component of the sample 34 or the two or more compositional components of the sample. The spectroscopy can be selected from the group consisting of absorption spectroscopy, fluorescence spectroscopy, X-ray spectroscopy, plasma emission spectroscopy, spark or arc (emission) spectroscopy, visible absorption spectroscopy, ultraviolet (UV) spectroscopy, infrared (IR) spectroscopy (including near-infrared (NIR) spectroscopy, mid-infrared (MIR) spectroscopy, and far-infrared (FIR) spectroscopy), Raman spectroscopy, coherent anti-Stokes Raman spectroscopy (CARS), nuclear magnetic resonance, photo emission, Mossbauer spectroscopy, acoustic spectroscopy, laser spectroscopy, Fourier transform spectroscopy, and Fourier transform infrared spectroscopy (FTIR) and combinations thereof. According to an embodiment, the spectroscopy method utilized is selected such that the at least one compositional component of the sample 34 is capable of being determined. According to another embodiment, the spectroscopy method utilized is selected such that two or more, and preferably a sufficient number of, compositional components of the sample 34 are capable of being determined.

The step of determining can include contacting the sample 34 with radiated energy. The analyzer 20 can include the source of radiated energy 22. The source of radiated energy can be ionizing radiation or non-ionizing radiation. The source of radiated energy 22 can be selected from the group consisting of a tunable source, a broadband source (BBS), a fiber amplified stimulated emission (ASE) source, black body radiation, enhanced black body radiation, a laser, infrared, supercontinuum radiation, frequency combined radiation, fluorescence, phosphorescence, and terahertz radiation. A broadband light source is a source containing the full spectrum of wavelengths, generally ranging from about 720 nm to about 1,620 nm. In an embodiment, the source of radiated energy 22 includes any type of infrared source.

The source of radiated energy 22 (e.g., light) can be emitted in a desired wavelength or range of wavelengths. The desired wavelength or range can be determined based on anticipated compositional components of the sample. According to an embodiment, the desired wavelength or range of wavelengths is selected such that the at least one compositional component of the sample can be determined. For example, in order to determine if CO₂ is present, the desired wavelength can be selected to be 4,300 nanometers (nm) as CO₂ has an absorption peak at that wavelength. The light emitted can also be in a range that encompasses the desired wavelength. For example, to detect CO₂, the light emitted can be in the mid-infrared range of approximately 2,500 to 25,000 nm. By way of another example, hydrogen sulfide gas (H₂S) can present absorption peaks at 1,900, 2,300, 2,600, 3,800 and 4,100 nm. According to this example the light emitted can include the entire IR spectrum or the NIR and MIR ranges of 800 to 2,500 nm and 2,500 to 25,000 nm, respectively. By way of another example, CH₄ can present an absorption peak at approximately 1,700 nm; whereas aromatics can present an absorption peak at approximately 2,450 nm. Accordingly, the light emitted can be in the near IR range.

According to an embodiment, the methods include the step of determining two or more compositional components of the sample 34. A separate analyzer 20, depicted as 20′ in the Figures, can be used for each compositional component to be determined. Of course each analyzer 20 can also be designed such that each analyzer is capable of determining the two or more compositional components of the sample 34. According to this embodiment, the wavelength or wavelength range can be selected such that the two or more compositional components of the sample 34 can be determined. By way of example, in order to determine if both CO₂ and H₂S are present in the sample, the wavelength range can be selected to be the MIR range of approximately 2,500 to 25,000 nm. In this manner, should CO₂ and

H₂S both be present in the sample, then absorption peaks would indicate such presence. By way of another example, in order to determine if both CH₄ and aromatics are present in the sample, the wavelength range can be selected to be the NIR range of approximately 800 to 2,500 nm. In an embodiment the source of radiated energy 22 is directed to the sample 34 in order to determine the two or more compositional components. The source of radiated energy 22 can transmit light rays in a range of 4,000 to 5,000 nm, which is a range for absorbance of carbon dioxide. The source of radiated energy 22 can also transmit light rays in a range of from 1,900 to 4,200 nm, which is a range for absorbance of hydrogen sulfide. Data collected from these two wavelength ranges may provide information for determining the presence of carbon dioxide and hydrogen sulfide in the sample 34.

The source of radiated energy 22 can be a light source. The light source can be in the IR range. According to an embodiment, the IR light source is a MIR range light source. In an embodiment the MIR range light source is a tunable light source. The tunable light source may be selected from the group of an optical parametric oscillator (OPO) pumped by a pulsed laser, a tunable laser diode, and a broadband source (BBS) with a tunable filter. In an embodiment, the tunable MIR light source is adapted to cause pulses of light to be emitted at or near the absorption peak of the at least one compositional component of the sample 34.

The water content of the sample can be determined in any manner and can be determined by optical or non-optical means. According to an embodiment, the water content in the sample and the compensation, if any, of the optical response shifts of the sample can be determined.

If the tunable light source is a broadband source, then detection of the at least one compositional component of the sample 34 may be improved by applying frequency modulation to the broadband source signal by modulating the drive current or by chopping so that unwanted signals can be avoided in the detector of the spectrometer by using phase sensitive detection. The broadband source may be pulsed with or without frequency modulation.

In an embodiment the source of radiated energy 22 can include a laser diode array. In a laser diode array light source system, desired wavelengths are generated by individual laser diodes. The output from the laser diode sources may be controlled in order to provide signals that are arranged together or in a multiplexed fashion. By utilizing a laser diode array light source, time and/or frequency division multiplexing may be accomplished at the spectrometer. A one-shot measurement or an equivalent measurement may be accomplished with the laser diode array. A probe-type or sample-type optical cell system may also be utilized.

The step of determining can further comprise detecting the interaction between the radiated energy and the sample 34. The detection of the interaction can occur via the use of at least one detector 24. According to an embodiment, the analyzer 20 can include at least one detector 24. If the analyzer 20 is a MOE calculation device, then the analyzer can further comprise a second detector (not shown). According to an embodiment, the detector 24 is capable of detecting the interaction between the radiated energy and the sample 34. The radiated energy can be partially or fully absorbed by the sample 34, wherein some or none of the radiated energy is then transmitted through the sample. According to an embodiment, the detector 24 is capable of detecting the amount of radiated energy that is absorbed and/or transmitted by the sample 34. The effectiveness of the detector 24 may be dependent upon temperature conditions. Generally, as temperatures increase, the detector 24 becomes less sensitive. The detector 24 can include a mechanism whereby thermal noise is reduced and sensitivity to emitted radiated energy is increased. The detector 24 can be selected from the group consisting of thermal piles, photo acoustic detectors, thermoelectric detectors, quantum dot detectors, momentum gate detectors, frequency combined detectors, high temperature solid gate detectors, and detectors enhanced by meta materials such as infinite index of refraction, and combinations thereof.

The source of radiated energy 22 can also include a splitter. For example, a light that is emitted can be split into two separate beams in which one beam passes through the sample 34 and the other beam passes through a reference sample. Both beams are subsequently directed to a splitter before passing to the detector 24. The splitter quickly alternates which of the two beams enters the detector. The two signals are then compared in order to determine the compositional component of the sample 34.

The spectroscopy can be performed by a diffraction grating or optical filter, which allows selection of different narrow-band wavelengths from a white light or broadband source. A broadband source can be used in conjunction with Fiber Bragg Grating (FBG). FBG includes a narrow band reflection mirror whose wavelength can be controlled by the FBG fabrication process. The broadband light source can be utilized in a fiber optic system. The fiber optic system can contain a fiber having a plurality of FBGs. Accordingly, the broadband source is effectively converted into a plurality of discrete sources having desired wavelengths.

The spectroscopy can also be Fourier spectroscopy. Fourier spectroscopy, or Fourier transform spectroscopy, is a method of measurement for collecting spectra. In Fourier transform spectroscopy, rather than allowing only one wavelength at a time to pass through the sample to the detector, this technique lets through a beam containing many different wavelengths of light at once, and measures the total beam intensity. Next, the beam is modified to contain a different combination of wavelengths, giving a second data point. This process is repeated many times. Afterwards, a computer takes all this data and works backwards to infer how much light there is at each wavelength. The analyzer 20 can include one or more mirrors used to select the desired wavelengths to pass through the sample 34 to the detector 24. There can be a certain configuration of mirrors that allows some wavelengths to pass through but blocks others (due to wave interference). The beam can be modified for each new data point by moving one of the mirrors; this changes the set of wavelengths that can pass through. The analyzer 20 can internally generate a fixed and variable length path for the optical beam and then recombine these beams, thereby generating optical interference. The resulting signal includes summed interference pattern for all wavelengths not absorbed by the sample. As a result, the measurement system is not a one-shot type system, and hence the sampler-type probe is preferred for use with this type of spectrometer.

The Fourier spectroscopy can utilize an IR light source, also referred to as Fourier transform infrared (FTIR) spectroscopy. In an embodiment, IR light is guided through an interferometer, the IR light then passes through the sample 34, and a measured signal is then obtained, called the interferogram. In an embodiment Fourier transform is performed on this signal data, which results in a spectrum identical to that from conventional infrared spectroscopy. The benefits of FTIR include a faster measurement of a single spectrum. The measurement is faster for the FTIR because the information at all wavelengths is detected simultaneously.

As can be seen in FIG. 2, the step of determining at least one compositional component of the sample can further comprise transmitting data from the detector 24 to a computer 12. The computer 12 can be used to analyze the data from the detector 24 such that the presence of one or more compositional components of the sample 34 can be determined. Either the raw detector data outputs may be sent to the computer 12, or the signals may be subtracted with an analog circuit and magnified with an operational amplifier converted to voltage and sent to the computer 12 as a proportional signal, for example.

As can be seen in FIGS. 2 and 3, the sample 34 may be located between the source of radiated energy 22 and the detector 24. As can be seen in FIG. 3, the analyzer can include a housing 26. The housing 26 can contain the source of radiated energy 22 and the detector 24. The housing 26 can be magnetized metal or stainless steel and may have appropriate protective coatings. The housing 26 can be circular, cylindrical, or rectangular. The housing 26 is preferably constructed so that it is readily attachable and detachable from a tube 72. The tube 72 preferably includes a circular or rectangular opening forming a window that is transparent to the radiated energy. In this manner, the radiated energy can penetrate through the opening and come in contact with the sample 34 flowing through the tube 72. The interaction between the radiated energy and the sample 34 can then be detected via the detector 24 and another opening in the tube 72 adjacent to the detector.

According to an embodiment, the step of determining is performed during the step of collecting the sample 34. The step of determining can be performed during fluid flow of the sample. The fluid flow can be during fluid flow of the sample 34 into the sample receptacle 30 during the step of collecting, or it can be during fluid flow of the sample from the sample receptacle into the second container 80. The following is one example of use according to this embodiment. The sample container 300 can be introduced into a well. As can be seen in FIG. 1, the analyzer 20 can be located at one end of the sample receptacle 30. One or more samples 34 can flow or be caused to flow through the tube 72, in fluid flow direction 54 and into one or more sample receptacles 30. As the one or more samples 34 flow into each sample receptacle 30, the analyzer 20 can be used to determine one or more compositional components of the fluid. The analyzer 20 determines the presence of the compositional component in real time and reports that information instantaneously as it occurs in the sample 34. Each sample container 300 can contain a plurality of sample receptacles 30. Moreover, there can be more than one sample container 300 and there can also be more than one analyzer 20. If there is more than one sample container 300, then a first analyzer 20 can be positioned adjacent to a first sample container 300 and a second analyzer 20′ can be positioned adjacent to a second sample container 300, etc. One analyzer 20 can be designed to determine a first compositional component of the sample 34, while another analyzer 20′ can be designed to determine a second compositional component of the sample 34.

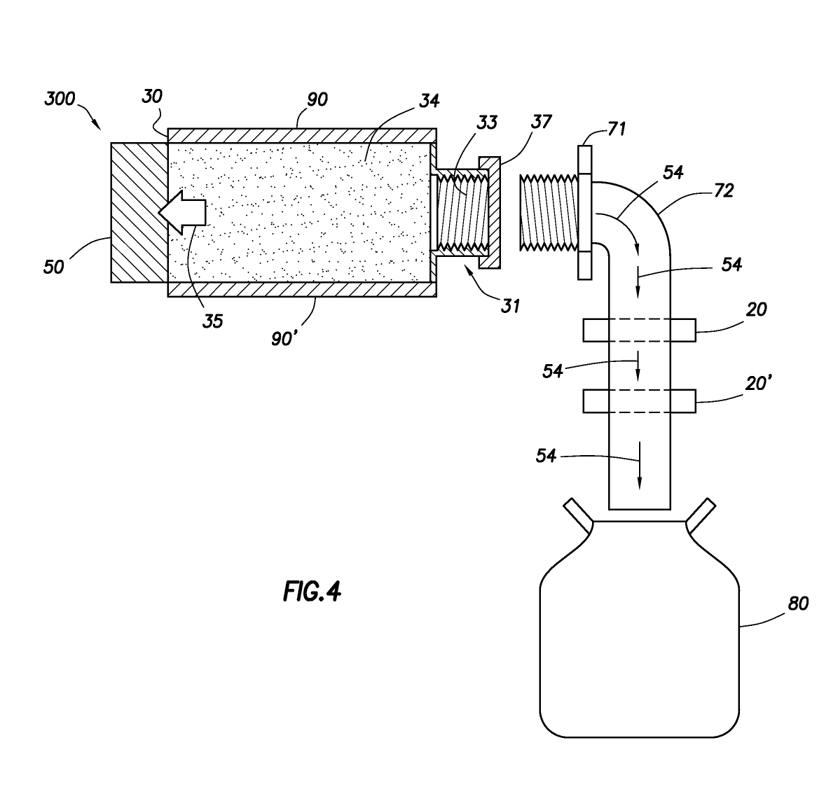

It is often desirable to transfer a collected sample into a storage container. The sample can be stored and/or transported off-site to another location for further analysis. According to an embodiment, and as can be seen in FIG. 4, the methods include the step of transferring the sample from the sample container 300 to a second container 80, wherein the step of transferring is performed after the step of collecting. The second container 80 can be a storage or transportation container. This may be desirable, for example, if the sample container 300 does not meet transportation regulations and the sample needs to be transported off-site. The sample 34 can be transferred via a tube 72. The tube 72 can be connected to the sample container 300 in a variety of ways, for example, in a manner such that the sample 34 is capable of being removed from the sample receptacle 30 and placed into the second container 80. By way of example, the sample container 300 can contain a female end 31 that is capable of connecting to a male end 71 of the tube 72. The ends can be threaded together, for example, via threads 33 on the female end 31. The female end 31 can also include a seal 37. The seal 37 can be removed prior to attaching the tube 72 to the sample container 300. The sample 34 can be transferred via a variety of means, for example, via a piston 50. This way, the sample 34 can flow from the sample receptacle 30, through the tube 72, and into the second container 80. The sample 34 can also be heated via one or more heating elements 90 and 90′.

According to an embodiment, the step of determining the at least one compositional component of the sample is performed after the step of collecting and is performed during fluid flow of the sample. The step of determining the at least one compositional component of the sample 34 can be performed during the step of transferring the sample 34 from the sample container 300 to the second container 80. One or more analyzers 20 and 20′ can be positioned adjacent to the tube 72. In this manner, as the sample 34 is being transferred from the sample receptacle 30 into the second container 80, the analyzer 20 can determine the at least one compositional component of the sample 34. As discussed above, a first analyzer 20 can be designed to determine a first compositional component of the sample 34 and a second analyzer 20′ can be designed to determine a second compositional component of the sample. Moreover, one analyzer 20 can also be designed to determine two or more compositional components of the sample. There can also be more than two analyzers 20 located adjacent to the tube 72.

The methods include the step of using an equation of state (EOS) to predict a potential change in at least one property of the reservoir fluid based on the determination of the at least one compositional component of the sample 34. According to another embodiment, two or more different EOS formulas can be used to predict the potential change. The step of using can be performed after the step of determining. The methods can further include the step of selecting the EOS to be used. The EOS can be selected from cubic or non-cubic equations of state. The EOS can be any EOS that can predict a potential change in the at least one property of the reservoir fluid. The EOS selected may vary depending on the one or more compositional components of the sample. According to an embodiment, the EOS is selected from the group consisting of Boyle, Van der Waals, Redlich-Kwong (RK), Soave-Redlich-Kwong (SRK), Peng-Robinson (PR), Peng-Robinson-Stryjek-Vera (PRSV), Patel-Teja (PT), Schmit-Wenzel (SW), and Esmaeilzadeh-Roshanfekr (ER).

The EOS is used to predict a potential change in at least one property of the reservoir fluid. The at least one property can be selected from the group consisting of weight, volume, mass, density, phase, composition, and combinations thereof. The methods can further include the step of inputting at least one state function into the EOS formula. The EOS can be calculated via the use of a software program. The step of inputting can include using a computer keyboard to input the state function(s) into the software program. The state function can be, without limitation, temperature, pressure, volume, density, composition, and combinations thereof. The methods can further include the step of solving the EOS using the at least one compositional component of the fluid and the input state function(s). By way of example, an investigator can input theoretical temperatures, pressures, etc., into the EOS formula. The EOS can then be solved and the software program can report the potential change in the at least one property of the fluid based on the input information (e.g., the compositional component and the state function).

The following is an example of a method of using data obtained from a sample of a reservoir fluid. Other examples could be given, and it is to be understood that this example is for illustrative purposes only and is not meant to limit the scope of the invention. A worker at a well site can collect a sample of the reservoir fluid. The worker can then determine at least one compositional component of the reservoir fluid using the analyzer. The worker can then select the EOS to be used. The worker can then input the data obtained from the analyzer as to the identity of the at least one compositional component. The worker can input at least one state function into the EOS formula. For example, if the worker wants to predict the behavior of the reservoir fluid (i.e., the potential change in at least one property of the fluid) if the temperature of the formation remains the same, but the pressure increases to a specific pressure, then the worker can input the temperature and specific pressure into the EOS formula. The EOS formula can then be solved to provide the predicted behavior of the fluid at the specific temperature and pressure. According to an embodiment, the EOS is used two or more times to predict a potential change in at least one property of the reservoir fluid. It is to be understood that at least one state function input will vary for each time the EOS is used. For example, a worker can input different state functions multiple times into the EOS. The predictions at each specific state function can give workers valuable insight into the potential behavior of the fluid over the life of the well and at many different wellbore conditions. In another embodiment, all the above steps can be undertaken automatically, namely with minimal instructions from knowledgeable personnel. The methods can be used to deliver an analytical interpretation of the fluid phase of a reservoir fluid, and the computer can then automatically provide the necessary interpretation and prediction of behavior changes to the fluid based on preset state functions.

The methods can further include the step of conducting one or more tuning tests on the sample 34. A tuning test can be used to yield more accurate predictions for a given EOS. The tuning test can be conducted on-site or off-site. The methods can further include the step of conducting at least one tuning test on the sample. The methods can further include the step of transporting the sample to an off-site laboratory, wherein the at least one tuning test is performed at the laboratory. The tuning test can be any test wherein the results from the tuning test will generate more accurate EOS predictions compared to an EOS without the results from the tuning test. The tuning test can be, without limitation, a single stage flash test or a constant composition expansion test, or any other physical measurement wherein the interpretation is used to tune the response of the EOS. For example, the tuning test can determine, among other things, the gas to oil ratio (GOR), the formation volume factor (FVF), the relative density of the dead oil (API gravity), bubble point, dew point, saturation pressure, and the flashed oil and gas compositions of the reservoir fluid sample.

The tuning test may not need to be performed for all reservoir fluids. For example, whether a tuning test should be conducted can depend on how many of the compositional components of the fluid are determined. For heavy crude oil, an EOS can yield accurate predictions when only 6 or 7 compositional components have been determined. However, as the fluid goes from heavy crude to light crude to liquid gas mixtures to gas condensates, then more compositional components need to be determined in order to obtain accurate predictions. If a fewer number of compositional components are available, then a tuning test can be performed in order to increase the accuracy of the EOS. For example, if the reservoir fluid sample is a gas condensate and 8 compositional components have been determined, then it may be necessary to conduct at least one tuning test on the sample in order to improve the accuracy of the EOS.

The methods can further include the step of transporting one or more of the samples off-site, wherein the step of transporting can be performed after the step of determining. The results from the analyzer 20 can be useful in deciding which, if any, of the samples might need to be transported off-site. By being able to determine at least one compositional component of the sample, workers are able to more accurately determine which samples may require further testing at an off-site location.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention. While compositions and methods are described in terms of “comprising,” “containing,” or “including” various components or steps, the compositions and methods also can “consist essentially of” or “consist of” the various components and steps. Whenever a numerical range with a lower limit and an upper limit is disclosed, any number and any included range falling within the range is specifically disclosed. In particular, every range of values (of the form, “from about a to about b,” or, equivalently, “from approximately a to b,”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

What is claimed is:

1. A method of using data obtained from a sample of a reservoir fluid comprising:
collecting the sample in a sample container,
wherein the sample container includes a sample receptacle, and
wherein the step of collecting comprises allowing or causing the sample to flow into the sample receptacle;
determining at least one compositional component of the sample using an analyzer, wherein the step of determining is performed during the step of collecting; and
using an equation of state to predict a potential change in at least one property of the reservoir fluid based on the determination of the at least one compositional component of the sample.

2. The method according to claim 1, wherein the step of determining is performed during fluid flow of the sample.

3. The method according to claim 1, wherein the at least one compositional component is selected from the group consisting of: asphaltenes; saturates; resins; aromatics; solid particulate content; hydrocarbon composition and content; gas composition carbon 1 to carbon 13 (C₁-C₁₃) and content; carbon dioxide gas; hydrogen sulfide gas; total stream percentage of water, gas, oil, and solid particles; water elements including ion composition and content, anions, cations, salinity, organics, contamination; or other hydrocarbon, gas, solids, or water properties that can be related to spectral characteristics, including the use of regression methods.

4. The method according to claim 1, wherein the number of compositional components of the sample determined is in the range of about 6 to greater than 20.

5. The method according to claim 1, further comprising the step of selecting the equation of state to be used.

6. The method according to claim 5, wherein at least a sufficient number of compositional components are determined such that the exact equation of state to be used can be selected.

7. The method according to claim 1, wherein the equation of state is selected from the group consisting of Boyle, Van der Waals, Redlich-Kwong (RK), Soave-Redlich-Kwong (SRK), Peng-Robinson (PR), Peng-Robinson-Stryjek-Vera (PRSV), Patel-Teja (PT), Schmit-Wenzel (SW), and Esmaeilzadeh-Roshanfekr (ER).

8. The method according to claim 1, wherein the at least one property is selected from the group consisting of weight, volume, mass, density, phase, composition, and combinations thereof.

9. The method according to claim 1, further comprising the step of inputting at least one state function into the equation of state.

10. The method according to claim 9, wherein the state function is selected from the group consisting of temperature, pressure, volume, density, composition, and combinations thereof.

11. The method according to claim 10, further comprising the step of solving the equation of state using the at least one compositional component of the fluid and the input state function.

12. A method of using data obtained from a sample of a reservoir fluid comprising:
collecting the sample in a sample container;
transferring the sample from the sample container to a second container, wherein the step of transferring is performed after the step of collecting;
determining at least one compositional component of the sample using an analyzer, wherein the step of determining is performed after the step of collecting; and
using an equation of state to predict a potential change in at least one property of the reservoir fluid based on the determination of the at least one compositional component of the sample.

13. The method according to claim 12, wherein the step of determining the at least one compositional component of the sample is performed during the step of transferring the sample from the sample container to the second container.

14. The method according to claim 12, wherein the step of determining is performed during fluid flow of the sample.

15. The method according to claim 12, wherein the at least one compositional component is selected from the group consisting of: asphaltenes; saturates; resins; aromatics; solid particulate content; hydrocarbon composition and content; gas composition carbon 1 to carbon 13 (C₁-C₁₃) and content; carbon dioxide gas; hydrogen sulfide gas; total stream percentage of water, gas, oil, and solid particles; water elements including ion composition and content, anions, cations, salinity, organics, contamination; or other hydrocarbon, gas, solids, or water properties that can be related to spectral characteristics, including the use of regression methods.

16. The method according to claim 12, wherein the number of compositional components of the sample determined is in the range of about 6 to greater than 20.

17. The method according to claim 12, further comprising the step of selecting the equation of state to be used.

18. The method according to claim 17, wherein at least a sufficient number of compositional components are determined such that the exact equation of state to be used can be selected.

19. The method according to claim 12, wherein the EOS is selected from the group consisting of Boyle, Van der Waals, Redlich-Kwong (RK), Soave-Redlich-Kwong (SRK), Peng-Robinson (PR), Peng-Robinson-Stryjek-Vera (PRSV), Patel-Teja (PT), Schmit-Wenzel (SW), and Esmaeilzadeh-Roshanfekr (ER).

20. The method according to claim 12, wherein the at least one property is selected from the group consisting of weight, volume, mass, density, phase, composition, and combinations thereof.

21. The method according to claim 12, further comprising the step of inputting at least one state function into the equation of state.

22. The method according to claim 21, wherein the state function is selected from the group consisting of temperature, pressure, volume, density, composition, and combinations thereof.

23. The method according to claim 22, further comprising the step of solving the equation of state using the at least one compositional component of the fluid and the input state function.
